(19) United States
(12) Patent Application Publication
(10) Pub. No.: US 2025/0241818 A1
(43) Pub. Date: Jul. 31, 2025

(54) LAYERED BAND FOR INCORPORATION INTO WEARABLE MEDICAL DEVICES

(71) Applicants: Stephen H. LIU, Los Angeles, CA (US); IFGCURE HOLDINGS, LLC, Los Angeles, CA (US)

(72) Inventor: Stephen H. LIU, Los Angeles, CA (US)

(21) Appl. No.: 18/854,665

(22) PCT Filed: Apr. 10, 2023
(86) PCT No.: PCT/US2023/018022

§ 371 Date: Oct. 7, 2024

Related U.S. Application Data
(60) Provisional application No. 63/329,305, filed on Apr. 8, 2022.

(51) Int. Cl. A61H 11/00; A41D 31/04

(52) U.S. Cl. CPC A61H 11/00; A41D 31/04; A61H 2011/005; A61H 2201/1253; A61H 2201/1619; A61H 2201/164; A61H 2201/165; A61H 2201/1695

(57) ABSTRACT

The present disclosure is directed to a layered band for incorporating into wearable medical device, wherein the band works to simultaneously activate, tighten, and elongate muscles. Embodiments of the present disclosure include pairs of men's and women's shorts incorporating the layered band in the waistband.

### FIELD OF THE DISCLOSURE

The present disclosure is related to the field of medical devices, especially a layered band for incorporation into wearable medical devices for lower body posture correction, improved ergonomics, and tension, activation, and elongation of abdominal, lumbar spine, and/or lower body muscles.

### BACKGROUND OF THE DISCLOSURE

One way the body adapts to static and dynamic positioning is through proprioception. Two proprioceptors that play an important role in the flexibility of muscles are the muscle spindles and the golgi tendon organs (GTOs), which work together to regulate muscle stiffness and activation. Muscle spindles sense muscle length while GTOs sense muscle tension. When a muscle spindle senses that its agonist muscle is being stretched too far, it will send an afferent signal to the brain that the muscle needs to contract as a protective mechanism. The brain receives this afferent signal and sends an efferent signal back to the agonist muscle to activate and produce contraction. The muscle spindle can also send an afferent signal to the brain to inhibit an antagonist (i.e., opposing) muscle so that it will relax and not contribute to any further stretching of the agonist muscle. GTOs work opposite of muscle spindles to relax an agonist muscle when there is too much tension. A GTO can sense tension in an agonist muscle either when the muscle is contracted or being stretched. GTOs not only sense tension in the muscle but also the rate of tension. When a muscle is stretched slowly, the GTO will sense the slow rate of tension and temporarily inhibit the muscle spindle so that the muscle can relax, which is what occurs during a static stretch. When the body is in a static position that causes stretch on the muscles, the muscle spindles and GTOs begin to adapt to new resting positions such that they will only send signals to the brain under circumstances that deviate from the adapted muscle lengths and tensions.

During dynamic movement, such as running, jumping, golfing, or athletic activity, the GTOs may sense a much higher rate of tension than during static positioning, so they do not inhibit the muscle spindles in the same way described above. Muscle spindles may exhibit a dynamic response to stretch by increasing afferent firing rates to contract the stretched muscles, which can be counterproductive to the intended movement. For example, if the adductors are told to contract when they are being stretched by a dynamic movement, then this can cause internal rotation of the femurs, closing the hips.

Accordingly, there exists a need in the art for a simple device that can be comfortably worn as normal clothing or under normal clothing (for example, at work, during athletic activities, while training, during rehabilitation, for muscle recovery, or on a road trip) that trains the body to proprioceptively achieve better posture, a neutral pelvis, and activation and elongation of the muscles the device interacts with. Accordingly, the present disclosure is aimed at solving these and other problems discussed below.

### SUMMARY OF THE DISCLOSURE

Certain embodiments of the present disclosure relate to a layered band for incorporation into wearable medical devices, such as wearable tech or performance wearables, wherein the band works to simultaneously activate, tighten, and elongate muscles. More specifically, certain embodiments are directed at men's and women's pants or leggings. In some embodiments, the layered band comprises three layers.

In some embodiments, the outer layers of the layered band are configured to create tension and the middle layer of the layered band is configured zo create elongation. Furthermore, in some embodiments, the fabric of the layers in the layered band comprises 3D tensile-compression-elongation properties to engage muscle memory.

Muscle memory is biofeedback and neurological adaptation. Simply put, muscle memory is the act of committing a specific motor task into memory through repetition. While muscles themselves cannot remember movement, they are full of neurons attached to the nervous system that play a role in motor learning. Any movement requiring brain activity, even complicated ones, done enough times triggers recognizable pattern in the area of the brain responsible for motor skills. Thus, leading to a learned motion and additional strength and flexibility that will require no brainpower in the future.

It is an objective of embodiments of the present disclosure to use 3D muscle memory engagement to treat posture and related musculoskeletal disorders. For example, neck and back pain, spasm, and stiffness.

It is also an objective of embodiments of the present disclosure to compress a wearer's muscles while simultaneously elongating.

It is a further objective of embodiments of the present disclosure to activate the wearer's muscles.

It is yet another objective of embodiments of the present disclosure to correct a wearer's posture by training the wearer's muscles and proprioceptors.

These and other further features and advantages provided in this disclosure would be apparent to those skilled in the art from the following detailed description, taken together with the accompanying drawings.

### BRIEF DESCRIPTION OF THE DRAWINGS

FIG. 1 is a photograph showing the exterior of a medical device incorporating a layered band according to one embodiment of the disclosure.

FIG. 2 is an exploded view of a layered band according to one embodiment of the disclosure.

FIG. 3 is a photograph of a medical device incorporating a layered band in the waistband according to according to one embodiment of the disclosure in its normal, unstretched state.

FIG. 3 is a photograph of a medical device incorporating a layered band in the waistband according to according to one embodiment of the disclosure in a stretched state.

### DETAILED DESCRIPTION OF THE DISCLOSURE

Throughout this disclosure, the embodiments illustrated should be considered as exemplars, rather than as limitations on the present disclosure. As used herein, the term “invention,” “device,” “apparatus,” “method,” “disclosure,” “present invention,” “present device,” “present apparatus,” “present method,” or “present disclosure” refers to any one of the embodiments of the disclosure described herein, and any equivalents. Furthermore, reference to various features of the “invention,” “device,” “apparatus,” “method,” “disclosure,” “present invention,” “present device,” “present apparatus,” “present method,” or “present disclosure” throughout this document does not mean that all claimed embodiments or methods must include the reference features.

It is also understood that when an element or feature is referred to as being “on” or “adjacent” to another element or feature, it can be directly on or adjacent the other element or feature or intervening elements or features may also be present. In contrast, when an element is referred to as being “directly on” or extending “directly onto” another element, there are no intervening elements present. Additionally, it is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Furthermore, relative terms such as “inner,” “outer,” “upper,” “top,” “above,” “lower,” “bottom,” “beneath,” “below,” and similar terms, may be used herein to describe a relationship of one element to another. Terms such as “higher,” “lower,” “wider,” “narrower,” and similar terms, may be used herein to describe angular relationships. It is understood that these terms are intended to encompass different orientations of the elements or system in addition to the orientation depicted in the figures.

Although the terms first, second, third, etc., may be used herein to describe various elements, components, regions, and/or sections, these elements, components, regions, and/or sections should not be limited by these terms. These terms are only used to distinguish one element, component, region, or section from another. Thus, unless expressly stated otherwise, a first element, component, region, or section discussed below could be termed a second element, component, region, or section without departing from the teachings of the present disclosure. As used herein, the term “and/or” includes any and all combinations of one or more of the associated list items.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. For example, when the present specification refers to “an” assembly, it is understood that this language encompasses a single assembly or a plurality or array of assemblies. It is further understood that the terms “comprises,” “comprising,” “includes,” and/or “including” when used herein, specify the presence of stated features, integers, steps, operations, elements, and/or components, but do not preclude the presence or addition of one or more other features, integers, steps, operations, elements, components, and/or groups thereof.

Embodiments as described in the present disclosure can be described herein with reference to view illustrations, some of which are schematic in nature. As such, the actual thickness of elements can be different, and variations from the shapes of the some of the illustrations as a result, for example, of manufacturing techniques and/or tolerances are expected. Thus, the elements illustrated in the some of the figures are schematic in nature and their shapes are not intended to illustrate the precise shape of a region and are not intended to limit the scope of the disclosure.

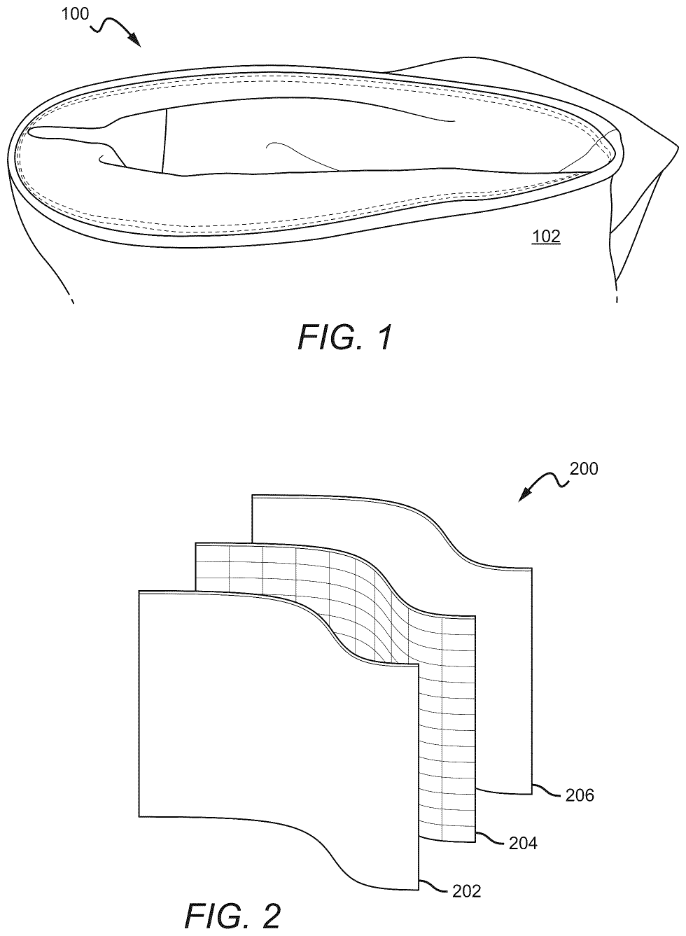

FIG. 1 is a photograph showing the exterior of a wearable medical device 100 incorporating the layered band according to one embodiment of the disclosure. As shown, the device 100 is a pair of women's leggings. In the embodiment shown, the device 100 comprises a waistband 102 and right and left legs. The waistband 102 shown incorporates the layered band disclosed herein. But, other embodiments of wearable tech incorporating the band disclosed herein are possible, for example, sports bras (e.g., a ribcage banc), shorts, wearable devices for joints (e.g., joint sleeves), and pants. Other embodiments of performance wearables may also incorporate the layered band disclosed herein in several locations throughout the device.

FIG. 2 shows an exploded view of a layered band incorporated into the device 100 shown in FIG. 1. As shown in FIG. 2, the layered band 200 may comprise three layers of fabric 202, 204, 206, although less or more layers are possible. In certain preferred embodiments, the layers 202, 204, 206 of the layered band 200 are affixed to one another. These layers may be affixed to one another by glue, stitching, or tape. It is understood many methods of affixing the layers to each other are possible.

In certain preferred embodiments, the middle fabric 204 comprises a mesh material that is able to elongate and/or pull the muscles that the layered band 200 interacts with. In certain preferred embodiments, the mesh material may further comprise a combination of nylon and spandex. It is understood that the layered band according to the present disclosure can be made of many different materials.

In certain preferred embodiments, the outer layers 202, 206 of the layered band 200 comprise a material that is able to compress the muscles that the layered band 200 interacts with. In certain preferred embodiments, the two outer layers of the layered band 200 comprises a combination of spandex and a high-resistance fabric.

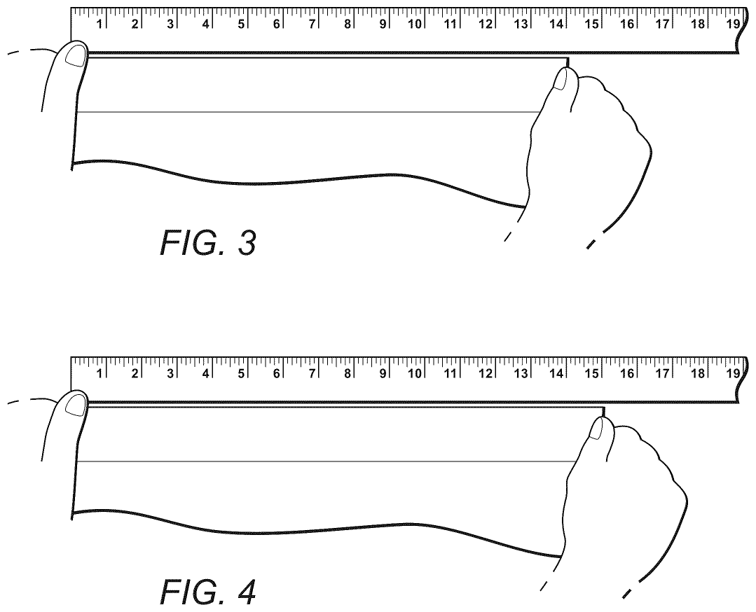

FIGS. 3 and 4 exemplify the tension effect of a layered band according to one embodiment. FIG. 3 shows a wearable medical device that incorporates a layered band according to the present disclosure in a relaxed, unstretched state. FIG. 4, alternatively, shows the same wearable medical device that incorporates a layered band according to the present disclosure in a tensed, stretched state. As can be seen in these figures, the layered band incorporated into wearable medical device prevents the device from stretching to great lengths.

The interaction between the layers 202, 204, 206 in the layered band 200 results in simultaneous tension, elongation, and activation of the muscles the layered band interacts with such as the abdominal, back, and certain lower body muscles. Tension, elongation, and activation of the muscles results in better posture, improved mobility, and aesthetic compression of a wearer's body. More specifically, the device 100 may help with dynamic positioning such as running or athletic activity.

It is understood that embodiments presented herein are meant to be exemplary. Embodiments of the present disclosure can comprise any combination or compatible features shown in the various figures, and these embodiments should not be limited to those expressly illustrated and discussed.

Although the present disclosure has been described in detail with reference to certain configurations thereof, other versions are possible. Further, none of the elements or features discussed herein should be construed as necessary, critical, or essential for any particular embodiment of the present disclosure. Therefore, the spirit and scope of the disclosure should not be limited to the versions described above. The foregoing is intended to cover all modifications and alternative constructions falling within the spirit and scope of the disclosure as expressed in the appended claims, wherein no portion of the disclosure is intended, expressly or implicitly, to be dedicated to the public domain if not set forth in the claims.

I claim:

1. A band for a wearable medical device, comprising:
first and second outer layers comprising a first material configured to compress a wearer's muscles; and
an inner layer interposed between said first and second outer layers, said inner layer comprising a second material configured to elongate a wearer's muscles.

2. The band of claim 1, wherein said first and second outer layers comprise spandex and high-resistance fabric.

3. The band of claim 1, wherein said inner layer comprises a mesh material.

4. The band of claim 1, wherein said inner layer comprises nylon and spandex.

5. The band of claim 1, wherein said first and second outer layers are affixed to said inner layer.

6. The band of claim 1, wherein said first and second outer layers are stitched to said inner layer.

7. The band of claim 1, wherein said band is configured as a waistband for pants.

8. The band of claim 1, wherein said band is configured as a ribcage band for bras.

9. The band of claim 1, wherein said band is configured as a joint sleeve.

10. A wearable medical device, comprising:
a garment; and
a band incorporated into said garment, said band comprising:
first and second outer layers comprising a first material configured to compress a wearer's muscles; and
an inner layer interposed between said first and second outer layers, said inner layer comprising a second material configured to elongate a wearer's muscles.

11. The wearable medical device of claim 10, wherein said first and second outer layers comprise spandex and high-resistance fabric.

12. The wearable medical device of claim 10, wherein said inner layer comprises a mesh material.

13. The wearable medical device of claim 10, wherein said inner layer comprises nylon and spandex.

14. The wearable medical device of claim 10, wherein said garment is a pair of pants or shorts.

15. The wearable medical device of claim 10, wherein said garment is a bra.
